(19) United States
(12) Patent Application Publication
(10) Pub. No.: US 2014/0171497 A1
(43) Pub. Date: Jun. 19, 2014

(54) ANIMAL SUPPLEMENTS AND COMPOSITIONS CONTAINING SOLUABLE MONENSIN AND METHODS THEREFOR

(71) Applicants: Jon Thomas Gawlak, Athens, AL (US); Steven Duff Lubetkin, Zionsville, IN (US)

(72) Inventors: Jon Thomas Gawlak, Athens, AL (US); Steven Duff Lubetkin, Zionsville, IN (US)

(73) Assignee: ELI LILLY AND COMPANY, Indianapolis, IN (US)

(21) Appl. No.: 14/232,691

(22) PCT Filed: Jul. 26, 2012
(86) PCT No.: PCT/US12/48303

§ 371 Date: Jan. 14, 2014

Related U.S. Application Data
(60) Provisional application No. 61/515,445, filed on Aug. 5, 2011.

(51) Int. Cl. A61K 31/351; A23K 1/18; A23K 1/17

(52) U.S. Cl. CPC A61K 31/351; A23K 1/17; A23K 1/1813
USPC 514/460

(57) ABSTRACT

The present disclosure provides animal feed supplements comprising a therapeutically effective amount of monensin and a solvent, wherein the supplement is a solution. The disclosure also provides food compositions comprising a therapeutically effective amount of monensin, a solvent, and an animal feed, methods of administering the food compositions, and processes for making the food compositions.

This application claims the benefit under 35 USC §119(e) of U.S. Provisional Application Ser. No. 61/515,445, filed on Aug. 5, 2011, the entire disclosure of which is incorporated herein by reference.

Monensin is an ionophore antibiotic isolated from the bacteria Streptomyces cinnamonensis. In veterinary medicine, monensin can be advantageously administered to animals for a variety of purposes. For example, administration of a therapeutically effective dose of monensin can be utilized for the treatment or prevention of ketosis and/or bloat, for the enhancement of milk production efficiency, for the enhancement of milk protein content in milk, for the enhancement of mineral uptake, for the enhancement of weight gain, for the enhancement of feed conversion efficiency, and the provision of desirable reproduction advantages.

Monensin is typically administered via a solid formulation that is ingested by an animal. For instance, a solid formulation of monensin can be a combined with a mineral mix (for example, a solid mineral mix) and/or an animal feed, followed by consumption of the combination by an animal. For bovines, the daily intake of monensin ingestion is targeted at about 50 milligrams (mg) to about 500 mg per head per day.

However, when a solid formulation of monensin is combined with a mineral mix and/or an animal feed, the amount of the combination ingested by an animal, particularly a bovine, is typically decreased compared to combinations that do not contain monensin. Without being bound by any theory, it is hypothesized that the solid formulation of monensin may have a taste which the animal prefers less than the monensin-free formulation. As a result, the animal may reduce its intake and ingestion of the monensin-containing combination. As hypothesized, the taste of the solid monensin formulation could be problematic to veterinary practice. For instance, the taste could decrease the intake of monensin itself and thus inhibit its therapeutic effectiveness. In addition, the taste could lower the consumption of the combined mineral mixes and feed, thus potentially contributing to myriad other problems in animals due to the decreased intake of nourishment.

Therefore, there exists a need for compositions and methods utilizing monensin that overcomes the limitations of its current solid formulations in order to benefit the intake of monensin, minerals, and feeds by animals. Accordingly, the present disclosure provides utilization of a soluble formulation of monensin that exhibits desirable properties and provides related advantages for its intake in animals.

The present disclosure demonstrates that the reduced intake of monensin can be overcome in animals by utilizing a soluble formulation comprising monensin and a solvent. By combining the soluble formulation of monensin with a mineral mix and/or an animal feed, the present invention overcomes the potential limitations of monensin's taste when used as a solid formulation.

The present disclosure provides animal feed supplements comprising a therapeutically effective amount of monensin and a solvent, wherein the supplement is a solution. The disclosure also provides food compositions comprising a therapeutically effective amount of monensin, a solvent, and an animal feed, methods of administering the food compositions, and processes for making the food compositions.

The monensin-containing animal feed supplements and food compositions according to the present disclosure provide several advantages compared to supplements and compositions utilizing a solid monensin formulation. First, the monensin-containing supplements and compositions of the present disclosure allow for increased intake of monensin by animals compared to products using a solid monensin formulation. Second, the ingestion of mineral mixes and animal feeds by animals are may also be increased compared to those using a solid monensin formulation. These advantages could be the result of the hypothesized improved taste of the monensin-containing animal feed supplements and food compositions according to the present disclosure.

Third, the monensin-containing compositions of the present disclosure are easier to mix homogenously, thus resulting in better spreading and minimized segregation of the product compared to those using a solid monensin formulation. Fourth, the monensin-containing supplements of the present disclosure may be prepared as solutions without the requirement of complex additional ingredients and can be easily combined with a mineral mix and/or an animal feed. Finally, the monensin-containing compositions of the present disclosure can be prepared easily and inexpensively compared to preparations using the solid monensin formulation, and could potentially be prepared on-site at the place of consumption.

Various embodiments of the present application utilize an animal feed supplement containing monensin and a solvent, wherein the supplement is a solution. As used herein, the term “solution” refers to a substantially homogenous one-phase system of two or more substances, for example a solute and a solvent. As used herein, the term “solute” refers to the dissolving phase of a solution. In some embodiments, the solute is monensin.

As used herein, the term “solvent” refers to the dispersing medium of a solution. In some embodiments, the solvent is a liquid at standard temperature and pressure, and one capable of solubilizing an appreciable amount of a specified solid solute. Solids vary from 0-100% in their degree of solubility. See, e.g., “Solubility Parameters of Organic Compounds,” CRC Handbook of Chemistry and Physics, 62d ed., C-699, CRC Press; N. Irving Sax and Richard J. Lewis, Sr., Hawley's Condensed Chemical Dictionary, 11th ed., 1079 (1987). The term “solvent” also includes combinations of two or more solvents.

As used herein, the term “monensin” refers to monensin base, pharmaceutically acceptable salts of monensin, or other salts of monensin. The term “pharmaceutically acceptable salt” refers to an addition salt that exists in conjunction with the acidic or basic portion of monensin. Such salts include the pharmaceutically acceptable salts listed in HANDBOOK OF PHARMACEUTICAL SALTS: PROPERTIES, SELECTION AND USE, P. H. Stahl and C. G. Wermuth (Eds.), Wiley-VCH, New York, 2002 which are known to the skilled artisan. In some embodiments, “monensin” is monensin sodium.

Pharmaceutically acceptable salts of an acid addition nature are formed when monensin and any of its intermediates containing a basic functionality are reacted with a pharmaceutically acceptable acid. Pharmaceutically acceptable acids commonly employed to form such acid addition salts include inorganic and organic acids. Pharmaceutically acceptable salts of a base addition nature are formed when monensin and any of its intermediates containing an acidic functionality are reacted with a pharmaceutically acceptable base. Pharmaceutically acceptable bases commonly employed to form base addition salts include organic and inorganic bases.

In addition to pharmaceutically acceptable salts, other salts are included in the present invention. They may serve as intermediates in the purification of compounds or in the preparation of other pharmaceutically-acceptable salts, or are useful for identification, characterization or purification.

Solvents according to the present disclosure are pharmaceutically acceptable for ingestion by an animal. A number of pharmaceutically acceptable solvents are known in the art. In some embodiments, the solvent is selected from the group consisting of benzyl alcohol, oleic acid, propylene glycol, vitamin E, ethanol, glyceryl mono- and di-caprylate, and mixtures thereof. In other embodiments, the solvent is benzyl alcohol. In other embodiments, the solvent is oleic acid. In other embodiments, the solvent is a derivatized propylene glycol (e.g., propylene glycol monocaprylate (Capryol 90™, Gattefossé Canada Inc). In other embodiments, the solvent is vitamin E. In other embodiments, the solvent is ethanol. In other embodiments, the solvent is glyceryl mono- and di-caprylate (i.e., Capmul MCM™, ABITEC Corporation).

The amount of monensin in the supplement is adequate to achieve a therapeutic effect. As used herein, the term “therapeutically effective amount” refers to an amount which gives the desired benefit to an animal and includes both treatment and prophylactic administration. The amount will vary from one individual to another and will depend upon a number of factors, including the overall physical condition of the animal and the underlying cause of the condition to be treated. The amount of monensin used for therapy gives an acceptable rate of change and maintains desired response at a beneficial level. A therapeutically effective amount of the present supplements may be readily ascertained by one of ordinary skill in the art using publicly available materials and procedures.

In some embodiments of the present disclosure, the amount of monensin in the supplement can vary. For example, in some embodiments, the amount of monensin can be present in the supplement in an amount of between about 5% to about 40% (weight of monensin/(weight of monensin plus solvent)). In some embodiments, the amount of monensin can be present in the supplement in an amount of between about 5% to about 35% (weight of monensin/(weight of monensin plus solvent)). In some embodiments, the amount of monensin can be present in the supplement in an amount of between about 5% to about 30% (weight of monensin/(weight of monensin plus solvent)). In some embodiments, the amount of monensin can be present in the supplement in an amount of between about 5% to about 25% (weight of monensin/(weight of monensin plus solvent)). In some embodiments, the amount of monensin can be present in the supplement in an amount of between about 5% to about 20% (weight of monensin/(weight of monensin plus solvent)). In some embodiments, the amount of monensin can be present in the supplement in an amount of between about 10% to about 20% (weight of monensin/(weight of monensin plus solvent)). In some embodiments, the amount of monensin is present in the supplement in an amount of about 25% (weight of monensin/(weight of monensin plus solvent)). In some embodiments, the amount of monensin is present in the supplement in an amount of about 20% (weight of monensin/(weight of monensin plus solvent)). In some embodiments, the amount of monensin is present in the supplement in an amount of about 15% (weight of monensin/(weight of monensin plus solvent)). In some embodiments, the amount of monensin is present in the supplement in an amount of about 10% (weight of monensin/(weight of monensin plus solvent)). In some embodiments, the amount of monensin is present in the supplement in an amount of about 5% (weight of monensin/(weight of monensin plus solvent)).

The amount of monensin in the supplement is a sufficient amount to provide animals, such as bovines, with between about 50 mg to about 500 mg per head per day. In some embodiments, the amount of monensin in the supplement provides animals with between about 100 mg of monensin to about 400 mg of monensin per day. In some embodiments, the amount of monensin in the supplement provides animals with about 100 mg of monensin per day. In some embodiments, the amount of monensin in the supplement provides animals with about 200 mg of monensin per day. In some embodiments, the amount of monensin in the supplement provides animals with about 250 mg of monensin per day. In some embodiments, the amount of monensin in the supplement provides animals with about 300 mg of monensin per day.

In some embodiments of the present disclosure, the amount of solvent in the supplement can vary. For example, in some embodiments, the amount of solvent can be present in the supplement in an amount of between about 60% to about 95% (weight of solvent/(weight of solvent plus monensin)). For example, in some embodiments, the amount of solvent can be present in the supplement in an amount of between about 70% to about 95% (weight of solvent/(weight of solvent plus monensin)). For example, in some embodiments, the amount of solvent can be present in the supplement in an amount of between about 75% to about 95% (weight of solvent/(weight of solvent plus monensin)). For example, in some embodiments, the amount of solvent can be present in the supplement in an amount of between about 80% to about 95% (weight of solvent/(weight of solvent plus monensin)). In some embodiments, the amount of solvent can be present in the supplement in an amount of between about 80% to about 90% (weight of solvent/(weight of solvent plus monensin)). In some embodiments, the amount of solvent is present in the supplement in an amount of 75% (weight of solvent/(weight of solvent plus monensin)). In some embodiments, the amount of solvent is present in the supplement in an amount of 80% (weight of solvent/(weight of solvent plus monensin)). In some embodiments, the amount of solvent is present in the supplement in an amount of 85% (weight of solvent/(weight of solvent plus monensin)). In some embodiments, the amount of solvent is present in the supplement in an amount of 90% (weight of solvent/(weight of solvent plus monensin)). In some embodiments, the amount of solvent is present in the supplement in an amount of 95% (weight of solvent/(weight of solvent plus monensin)).

In some embodiments described in the present disclosure, the supplements are associated with an improvement in consumption of the supplement. As used herein, the term “improvement in consumption” can refer to an increase in the amount of monensin consumed by an animal fed the supplement of the present disclosure compared to the amount of monensin consumed by an animal fed a solid formulation of monensin. In some embodiments, the supplement is consumed by an animal at a rate of about 50 mg of monensin to about 500 mg of monensin per day. In some embodiments, the supplement is consumed by an animal at a rate of about 100 mg of monensin to about 400 mg of monensin per day. In some embodiments, the supplement is consumed by an animal at a rate of about 100 mg of monensin per day. In some embodiments, the supplement is consumed by an animal at a rate of about 200 mg of monensin per day. In some embodiments, the supplement is consumed by an animal at a rate of about 250 mg of monensin per day. In some embodiments, the supplement is consumed by an animal at a rate of about 300 mg of monensin per day.

In another embodiment of the present disclosure, a food composition comprising a therapeutically effective amount of monensin, a solvent, and an animal feed is described. As used herein, the term “animal feed” refers to a composition that can be ingested by an animal, such as an animal feedstuff. The solvents, concentrations of monensin (i.e., weight of monensin/weight of monensin plus solvent), concentrations of solvent (i.e., weight of solvent/(weight of solvent plus monensin)), amounts of monensin, amounts of consumption, and improvement in consumption provided in the preceding paragraphs are also applicable to the food compositions described herein.

In some embodiments, the food composition further comprises a mineral mix. As used herein, the term “mineral mix” refers to a combination of at least one known mineral with at least one other known mineral. In one example, a mineral mix comprises sodium chloride, calcium carbonate, a carrier, and a number of minor nutrient species.

In some embodiments, the food composition contains a solvent wherein the solvent is present in a residual amount. As used herein, the term “residual” refers to the remaining portion of a solvent present in the food composition after an initial portion of the solvent has evaporated. For example, if the solvent is a relatively non-volatile solvent (e.g., benzyl alcohol), it will tend evaporate slowly after combination and may be present at a greater amount in the food composition. However, if the solvent is a relatively volatile solvent (e.g., ethanol), it will tend to evaporate quickly after combination and a residual amount will be present in the food composition. In some embodiments, a residual amount can be an amount of solvent that is about 0.1% to about 10% of the original amount of solvent initially present in the food composition. In other embodiments, a residual amount can be an amount of solvent that is about 0.1% to about 1% of the original amount of solvent initially present in the food composition. In other embodiments, a residual amount can be an amount of solvent that is about 1% to about 8% of the original amount of solvent initially present in the food composition. In other embodiments, a residual amount can be an amount of solvent that is about 4% to about 6% of the original amount of solvent initially present in the food composition. In other embodiments, a residual amount can be an amount of solvent that is about 5% of the original amount of solvent initially present in the food composition. In other embodiments, a residual amount can be an amount of solvent that is about 2.5% of the original amount of solvent initially present in the food composition. In other embodiments, a residual amount can be an amount of solvent that is about 1% of the original amount of solvent initially present in the food composition. In other embodiments, a residual amount can be an amount of solvent that is about 0.1% of the original amount of solvent initially present in the food composition. In other embodiments, a residual amount can be an amount of solvent that is less than 0.1% of the original amount of solvent initially present in the food composition.

In other embodiments described in the present disclosure, various methods comprising administering to an animal in need thereof a food composition comprising a therapeutically effective amount of monensin, a solvent, and an animal feed are described. As used herein, the term “administered” is used in its broadest sense and refers to any method of delivering a substance to an animal. In some embodiments, the food composition is administered to an animal via consumption by the animal. As used herein, the term “consumption” refers to the intake or ingestion of a substance by an animal, for example by eating the substance. The solvents, concentrations of monensin (i.e., weight of monensin/(weight of monensin plus solvent)), concentrations of solvent (i.e., weight of solvent/(weight of solvent plus monensin)), amounts of monensin, amounts of consumption, and improvement in consumption provided in the preceding paragraphs are also applicable to the methods described herein.

In some embodiments, a method of improving feed efficiency is described. As used herein, the term “improving feed efficiency” refers to an improvement in the ratio of unit of feed/forage consumed to unit of animal weight gain (i.e., unit of feed/forage consumed:unit of animal weight gain) over a specific time period.

In some embodiments, a method of increasing milk production efficiency is described. As used herein, the term “increasing milk production efficiency” refers to an increase in animal production of marketable solids per unit of feed intake.

In some embodiments, a method of increasing rate of weight gain is described. As used herein, the term “increasing rate of weight gain” refers to an increase in the ratio of unit of animal weight gain to unit of time (i.e., unit of animal weight gain:unit of time) over a specific time period.

In some embodiments, a method of preventing or treating of coccidiosis is described. “Preventing” refers to reducing the likelihood that the patient will incur or develop any of the pathological conditions described herein and includes prophylactic administration. The term “preventing” is particularly applicable to a patient that is susceptible to the particular pathological condition. “Treating” refers to mediating a disease or condition and preventing, reversing the clinical effects of the disease, mitigating its further progression, or ameliorating the symptoms associated with the disease or condition.

As used herein, the term “coccidiosis” refers to a parasitic disease of the intestinal tract caused by protozoans. In some embodiments, the coccidiosis is caused by a species from the genus Eimeria. In one embodiment, the coccidiosis is caused by Eimeria bovis. In one embodiment, the coccidiosis is caused by Eimeria zuernii. In one embodiment, the coccidiosis is caused by Eimeria crandallis. In one embodiment, the coccidiosis is caused by Eimeria christenseni. In one embodiment, the coccidiosis is caused by Eimeria ninakohlyakimovae.

In some embodiments, the food composition is administered to a ruminant. As used herein, the term “ruminant” refers to an even-toed hoofed animal that has a complex 3-chamber or 4-chamber stomach and which typically re-chews what it has previously swallowed. Some non-exhaustive examples of ruminants include bovines, sheep, goats, oxen, muskox, llamas, alpacas, guanicos, deer, bison, antelopes, camels, and giraffes. In one embodiment, the ruminant is a bovine. In another embodiment, the ruminant is a goat.

In some embodiments, the food composition is administered to an avian. As used herein, the term “avian” refers to a warm-blooded, egg laying, feathered vertebrate provided with wings, for example birds of any known species or type. In some embodiments, avians include poultry. As used herein, the term “poultry” means any domestic fowl reared for the table, or their eggs or feathers including chickens, (for example, White Leghorn, Brown Leghorn, Barred-Rock, Sussex, N.H., Rhode Island, Ausstralorp, Minorca, Amrox, Calif. Gray, Italian Partidge-colored, etc.), broilers, fryers, cocks and hens, capons, turkeys, ducks, geese, pheasants, quails, ostriches and other poultry commonly bred in commercial quantities.

In other embodiments described in the present disclosure, a process for preparing a food composition comprising combining a) a solution comprising monensin and a solvent, and b) an animal feed is described. In some embodiments, the process further comprises combining a) and b) with c) a mineral mix. The solvents, concentrations of monensin (i.e., weight of monensin/(weight of monensin plus solvent)), concentrations of solvent (i.e., weight of solvent/(weight of solvent plus monensin)), amounts of monensin, amounts of consumption, and improvement in consumption provided in the preceding paragraphs are also applicable to the processes described herein.

The following embodiments are also contemplated:
1. An animal feed supplement comprising a therapeutically effective amount of monensin and a solvent, wherein the supplement is a solution.
2. The animal feed supplement of clause 1 wherein the solvent is selected from the group consisting of benzyl alcohol, oleic acid, propylene glycol, vitamin E, ethanol, glyceryl mono- and di-caprylate, and mixtures thereof
3. The animal feed supplement of clause 1 or clause 2 wherein the solvent is benzyl alcohol.
4. The animal feed supplement of clause 1 or clause 2 wherein the solvent is oleic acid.
5. The animal feed supplement of clause 1 or clause 2 wherein the solvent is propylene glycol.
6. The animal feed supplement of clause 1 or clause 2 wherein the solvent is vitamin E.
7. The animal feed supplement of clause 1 or clause 2 wherein the solvent is ethanol.
8. The animal feed supplement of clause 1 or clause 2 wherein the solvent is glyceryl mono- and di-caprylate
9. The animal feed supplement of any of clauses 1 to 8 wherein monensin is present at a concentration of about 5% to about 40% (weight/weight) of said solution.
10. The animal feed supplement of any of clauses 1 to 9 wherein monensin is present at a concentration of about 5% to about 35% (weight/weight) of said solution.
11. The animal feed supplement of any of clauses 1 to 10 wherein monensin is present at a concentration of about 5% to about 30% (weight/weight) of said solution.
12. The animal feed supplement of any of clauses 1 to 11 wherein monensin is present at a concentration of about 5% to about 25% (weight/weight) of said solution.
13. The animal feed supplement of any of clauses 1 to 12 wherein monensin is present at a concentration of about 5% to about 20% (weight/weight) of said solution.
14. The animal feed supplement of any of clauses 1 to 13 wherein monensin is present at a concentration of about 10% to about 20% (weight/weight) of said solution.
15. The animal feed supplement of any of clauses 1 to 12 wherein monensin is present at a concentration of approximately 25% (weight/weight) of said solution.
16. The animal feed supplement of any of clauses 1 to 14 wherein monensin is present at a concentration of approximately 20% (weight/weight) of said solution.
17. The animal feed supplement of any of clauses 1 to 14 wherein monensin is present at a concentration of approximately 15% (weight/weight) of said solution.
18. The animal feed supplement of any of clauses 1 to 14 wherein monensin is present at a concentration of approximately 10% (weight/weight) of said solution.
19. The animal feed supplement of any of clauses 1 to 13 wherein monensin is present at a concentration of approximately 5% (weight/weight) of said solution.
20. The animal feed supplement of any of clauses 1 to 19 wherein the solvent is present at a concentration of about 60% to about 95% (weight/weight) of said solution.
21. The animal feed supplement of any of clauses 1 to 20 wherein the solvent is present at a concentration of about 70% to about 95% (weight/weight) of said solution.
22. The animal feed supplement of any of clauses 1 to 21 wherein the solvent is present at a concentration of about 75% to about 95% (weight/weight) of said solution.
23. The animal feed supplement of any of clauses 1 to 14 or clauses 16 to 22 wherein the solvent is present at a concentration of about 80% to about 95% (weight/weight) of said solution.
24. The animal feed supplement of any of clauses 1 to 14 or clauses 16 to 23 wherein the solvent is present at a concentration of about 80% to about 90% (weight/weight) of said solution.
25. The animal feed supplement of any of clauses 1 to 14 or clauses 16 to 24 wherein the solvent is present at a concentration of approximately 80% (weight/weight) of said solution.
26. The animal feed supplement of any of clauses 1 to 14 or clauses 17 to 24 wherein the solvent is present at a concentration of approximately 85% (weight/weight) of said solution.
27. The animal feed supplement of any of clauses 1 to 14 or clauses 18 to 24 wherein the solvent is present at a concentration of approximately 90% (weight/weight) of said solution.
28. The animal feed supplement of any of clauses 1 to 14 or clauses 19 to 23 wherein the solvent is present at a concentration of approximately 95% (weight/weight) of said solution.
29. The animal feed supplement of any of clauses 1 to 28 wherein the supplement is consumed by an animal at a rate of about 50 milligrams of monensin to about 500 milligrams of monensin per day.
30. The animal feed supplement of any of clauses 1 to 29 wherein the supplement is consumed by an animal at a rate of about 100 milligrams of monensin to about 400 milligrams of monensin per day.
31. The animal feed supplement of any of clauses 1 to 30 wherein the supplement is consumed by the animal at a rate of approximately 100 milligrams of monensin per day.
32. The animal feed supplement of any of clauses 1 to 30 wherein the supplement is consumed by the animal at a rate of approximately 200 milligrams of monensin per day.
33. The animal feed supplement of any of clauses 1 to 30 wherein the supplement is consumed by the animal at a rate of approximately 250 milligrams of monensin per day.
34. The animal feed supplement of any of clauses 1 to 30 wherein the supplement is consumed by the animal at a rate of approximately 300 milligrams of monensin per day.
35. The animal feed supplement of any of clauses 1 to 34 wherein the supplement is associated with an improvement in consumption by an animal.
36. A food composition comprising a therapeutically effective amount of monensin, a solvent, and an animal feed.
37. The food composition of clause 36 wherein the composition further comprises a mineral mix.
38. The food composition of clause 36 or clause 37 wherein the solvent is present in a residual amount.
39. The food composition of any one of clauses 36 to 38 wherein the solvent is selected from the group consisting of benzyl alcohol, oleic acid, propylene glycol, vitamin E, ethanol, glyceryl mono- and di-caprylate, and mixtures thereof
40. The food composition of any one of clauses 36 to 39 wherein the solvent is benzyl alcohol.
41. The food composition of any one of clauses 36 to 39 wherein the solvent is oleic acid.
42. The food composition of any one of clauses 36 to 39 wherein the solvent is propylene glycol.
43. The food composition of any one of clauses 36 to 39 wherein the solvent is vitamin E.
44. The food composition of any one of clauses 36 to 39 wherein the solvent is ethanol.
45. The food composition of any one of clauses 36 to 39 wherein the solvent is glyceryl mono- and di-caprylate.
46. The food composition of any one of clauses 36 to 45 wherein monensin is present at a concentration of about 5% to about 40% (weight/weight).
47. The food composition of any one of clauses 36 to 46 wherein monensin is present at a concentration of about 5% to about 35% (weight/weight).
48. The food composition of any one of clauses 36 to 47 wherein monensin is present at a concentration of about 5% to about 30% (weight/weight).
49. The food composition of any one of clauses 36 to 48 wherein monensin is present at a concentration of about 5% to about 25% (weight/weight).
50. The food composition of any one of clauses 36 to 49 wherein monensin is present at a concentration of about 5% to about 20% (weight/weight).
51. The food composition of any one of clauses 36 to 50 wherein monensin is present at a concentration of about 10% to about 20% (weight/weight).
52. The food composition of any one of clauses 36 to 49 wherein monensin is present at a concentration of approximately 25% (weight/weight).
53. The food composition of any one of clauses 36 to 51 wherein monensin is present at a concentration of approximately 20% (weight/weight).
54. The food composition of any one of clauses 36 to 51 wherein monensin is present at a concentration of approximately 15% (weight/weight).
55. The food composition of any one of clauses 36 to 51 wherein monensin is present at a concentration of approximately 10% (weight/weight).
56. The food composition of any one of clauses 36 to 50 wherein monensin is present at a concentration of approximately 5% (weight/weight).
57. The food composition of any one of clauses 36 to 56 wherein the solvent is present at a concentration of about 60% to about 95% (weight/weight).
58. The food composition of any one of clauses 36 to 57 wherein the solvent is present at a concentration of about 70% to about 95% (weight/weight).
59. The food composition of any one of clauses 36 to 58 wherein the solvent is present at a concentration of about 75% to about 95% (weight/weight).
60. The food composition of any one of clauses 36 to 59 wherein the solvent is present at a concentration of about 80% to about 95% (weight/weight).
61. The food composition of any one of clauses 36 to 51, clauses 53 to 55, or clauses 57 to 60 wherein the solvent is present at a concentration of about 80% to about 90% (weight/weight).
62. The food composition of any one of clauses 36 to 51 or clauses 53 to 61 wherein the solvent is present at a concentration of approximately 80% (weight/weight).
63. The food composition of any one of clauses 36 to 51 or clauses 54 to 61 wherein the solvent is present at a concentration of approximately 85% (weight/weight).
64. The food composition of any one of clauses 36 to 51 or clauses 55 to 61 wherein the solvent is present at a concentration of approximately 90% (weight/weight).
65. The food composition of any one of clauses 36 to 50 or clauses 56 to 60 wherein the solvent is present at a concentration of approximately 95% (weight/weight).
66. The food composition of any one of clauses 36 to 65 wherein the composition is consumed by an animal at a rate of about 50 milligrams of monensin to about 500 milligrams of monensin per day.
67. The food composition of any one of clauses 36 to 66 wherein the composition is consumed by an animal at a rate of about 100 milligrams of monensin to about 400 milligrams of monensin per day.
68. The food composition of any one of clauses 36 to 67 wherein the composition is consumed by an animal at a rate of approximately 100 milligrams of monensin per day.
69. The food composition of any one of clauses 36 to 67 wherein the composition is consumed by an animal at a rate of approximately 200 milligrams of monensin per day.
70. The food composition of any one of clauses 36 to 67 wherein the composition is consumed by an animal at a rate of approximately 250 milligrams of monensin per day.
71. The food composition of any one of clauses 36 to 67 wherein the composition is consumed by an animal at a rate of approximately 300 milligrams of monensin per day.
72. The food composition of any one of clauses 36 to 71 wherein the composition is associated with an improvement in consumption by an animal.
73. A method of improving feed efficiency comprising administering to an animal in need thereof a food composition comprising a therapeutically effective amount of monensin, a solvent, and an animal feed.
74. A method of increasing milk production efficiency comprising administering to an animal in need the thereof a food composition comprising a therapeutically effective amount of monensin, a solvent, and an animal feed.
75. A method of increasing rate of weight gain comprising administering to an animal in need thereof a food composition comprising a therapeutically effective amount of monensin, a solvent, and an animal feed.
76. A method of preventing or treating of coccidiosis comprising administering to an animal in need thereof a food composition comprising a therapeutically effective amount of monensin, a solvent, and an animal feed.
77. The method of clause 76 wherein the coccidiosis is caused by a species of Eimeria.
78. The method of clause 76 or clause 77 wherein the coccidiosis is caused by Eimeria bovis.
79. The method of clause 76 or clause 77 wherein the coccidiosis is caused by Eimeria zuernii.
80. The method of clause 76 or clause 77 wherein the coccidiosis is caused by Eimeria crandallis.
81. The method of clause 76 or clause 77 wherein the coccidiosis is caused by Eimeria christenseni.
82. The method of clause 76 or clause 77 wherein the coccidiosis is caused by Eimeria ninakohlyakimovae.
83. The method of any one of clauses 73 to 82 wherein the composition further comprises a mineral mix.
84. The method of any one of clauses 73 to 83 wherein the solvent is present in a residual amount.
85. The method of any one of clauses 73 to 84 wherein the animal is a ruminant.
86. The method of clause 85 wherein the ruminant is a bovine.
87. The method of clause 85 wherein the ruminant is a goat.
88. The method of any one of clauses 73 to 84 wherein the animal is an avian.
89. The method of any one of clauses 73 to 88 wherein the solvent is selected from the group consisting of benzyl alcohol, oleic acid, propylene glycol, vitamin E, ethanol, glyceryl mono- and di-caprylate, and mixtures thereof
90. The method of any one of clauses 73 to 89 wherein the solvent is benzyl alcohol.
91. The method of any one of clauses 73 to 89 wherein the solvent is oleic acid.
92. The method of any one of clauses 73 to 89 wherein the solvent is propylene glycol.
93. The method of any one of clauses 73 to 89 wherein the solvent is vitamin E.
94. The method of any one of clauses 73 to 89 wherein the solvent is ethanol.
95. The method of any one of clauses 73 to 89 wherein the solvent is glyceryl mono- and di-caprylate.
96. The method of any one of clauses 73 to 95 wherein monensin is present at a concentration of about 5% to about 40% (weight/weight).
97. The method of any one of clauses 73 to 96 wherein monensin is present at a concentration of about 5% to about 35% (weight/weight).
98. The method of any one of clauses 73 to 97 wherein monensin is present at a concentration of about 5% to about 30% (weight/weight).
99. The method of any one of clauses 73 to 98 wherein monensin is present at a concentration of about 5% to about 25% (weight/weight).
100. The method of any one of clauses 73 to 99 wherein monensin is present at a concentration of about 5% to about 20% (weight/weight).
101. The method of any one of clauses 73 to 100 wherein monensin is present at a concentration of about 10% to about 20% (weight/weight).
102. The method of any one of clauses 73 to 99 wherein monensin is present at a concentration of approximately 25% (weight/weight).
103. The method of any one of clauses 73 to 101 wherein monensin is present at a concentration of approximately 20% (weight/weight).
104. The method of any one of clauses 73 to 101 wherein monensin is present at a concentration of approximately 15% (weight/weight).
105. The method of any one of clauses 73 to 101 wherein monensin is present at a concentration of approximately 10% (weight/weight).
106. The method of any one of clauses 73 to 100 wherein monensin is present at a concentration of approximately 5% (weight/weight).
107. The method of any one of clauses 73 to 106 wherein the solvent is present at a concentration of about 60% to about 95% (weight/weight).
108. The method of any one of clauses 73 to 107 wherein the solvent is present at a concentration of about 70% to about 95% (weight/weight).
109. The method of any one of clauses 73 to 108 wherein the solvent is present at a concentration of about 75% to about 95% (weight/weight).
110. The method of any one of clauses 73 to 101 or clauses 103 to 109 wherein the solvent is present at a concentration of about 80% to about 95% (weight/weight).
111. The method of any one of clauses 73 to 101 or clauses 103 to 105 or clauses 107 to 110 wherein the solvent is present at a concentration of about 80% to about 90% (weight/weight).
112. The method of any one of clauses 73 to 101 or clauses 103 to 111 wherein the solvent is present at a concentration of approximately 80% (weight/weight).
113. The method of any one of clauses 73 to 101 or clauses 104 to 111 wherein the solvent is present at a concentration of approximately 85% (weight/weight).
114. The method of any one of clauses 73 to 101 or clauses 105 to 111 wherein the solvent is present at a concentration of approximately 90% (weight/weight).
115. The method of any one of clauses 73 to 101 or clauses 106 to 110 wherein the solvent is present at a concentration of approximately 95% (weight/weight).
116. The method of any one of clauses 73 to 115 wherein the composition is consumed by an animal at a rate of about 50 milligrams of monensin to about 500 milligrams of monensin per day.
117. The method of any one of clauses 73 to 116 wherein the composition is consumed by an animal at a rate of about 100 milligrams of monensin to about 400 milligrams of monensin per day.
118. The method of any one of clauses 73 to 117 wherein the composition is consumed by an animal at a rate of approximately 100 milligrams of monensin per day.
119. The method of any one of clauses 73 to 117 wherein the composition is consumed by an animal at a rate of approximately 200 milligrams of monensin per day.
120. The method of any one of clauses 73 to 117 wherein the composition is consumed by an animal at a rate of approximately 250 milligrams of monensin per day.
121. The method of any one of clauses 73 to 117 wherein the composition is consumed by an animal at a rate of approximately 300 milligrams of monensin per day.
122. The method of any one of clauses 73 to 121 wherein the method is associated with an improvement in consumption of the composition by the animal.
123. A process for preparing a food composition comprising combining a) a solution comprising monensin and a solvent, and b) an animal feed.
124. The process of clause 123 further comprising combining with c) a mineral mix.
125. The process of clause 123 or 124 wherein the solvent is selected from the group consisting of benzyl alcohol, oleic acid, propylene glycol, vitamin E, ethanol, glyceryl mono- and di-caprylate, and mixtures thereof.
126. The process of any one of clauses 123 to 125 wherein the solvent is benzyl alcohol.
127. The process of any one of clauses 123 to 125 wherein the solvent is oleic acid.
128. The process of any one of clauses 123 to 125 wherein the solvent is propylene glycol.
129. The process of any one of clauses 123 to 125 wherein the solvent is vitamin E.
130. The process of any one of clauses 123 to 125 wherein the solvent is ethanol.
131. The process of any one of clauses 123 to 125 wherein the solvent is glyceryl mono- and di-caprylate.
132. The process of any one of clauses 123 to 131 wherein monensin is present at a concentration of about 5% to about 40% (weight/weight).
133. The process of any one of clauses 123 to 132 wherein monensin is present at a concentration of about 5% to about 35% (weight/weight).
134. The process of any one of clauses 123 to 133 wherein monensin is present at a concentration of about 5% to about 30% (weight/weight).
135. The process of any one of clauses 123 to 134 wherein monensin is present at a concentration of about 5% to about 25% (weight/weight).
136. The process of any one of clauses 123 to 135 wherein monensin is present at a concentration of about 5% to about 20% (weight/weight).
137. The process of any one of clauses 123 to 136 wherein monensin is present at a concentration of about 10% to about 20% (weight/weight).
138. The process of any one of clauses 123 to 135 wherein monensin is present at a concentration of approximately 25% (weight/weight).
139. The process of any one of clauses 123 to 137 wherein monensin is present at a concentration of approximately 20% (weight/weight).
140. The process of any one of clauses 123 to 137 wherein monensin is present at a concentration of approximately 15% (weight/weight).
141. The process of any one of clauses 123 to 137 wherein monensin is present at a concentration of approximately 10% (weight/weight).
142. The process of any one of clauses 123 to 136 wherein monensin is present at a concentration of approximately 5% (weight/weight).
143. The process of any one of clauses 123 to 142 wherein the solvent is present at a concentration of about 60% to about 95% (weight/weight).
144. The process of any one of clauses 123 to 143 wherein the solvent is present at a concentration of about 70% to about 95% (weight/weight).
145. The process of any one of clauses 123 to 144 wherein the solvent is present at a concentration of about 75% to about 95% (weight/weight).
146. The process of any one of clauses 123 to 137 or clauses 139 to 145 wherein the solvent is present at a concentration of about 80% to about 95% (weight/weight).
147. The process of any one of clauses 123 to 137 or clauses 139 to 146 wherein the solvent is present at a concentration of about 80% to about 90% (weight/weight).
148. The process of any one of clauses 123 to 137 or clauses 139 to 147 wherein the solvent is present at a concentration of approximately 80% (weight/weight).
149. The process of any one of clauses 123 to 137 or clauses 140 to 147 wherein the solvent is present at a concentration of approximately 85% (weight/weight).
150. The process of any one of clauses 123 to 137 or clauses 141 to 147 wherein the solvent is present at a concentration of approximately 90% (weight/weight).
151. The process of any one of clauses 123 to 137 or clauses 142 to 146 wherein the solvent is present at a concentration of approximately 95% (weight/weight).

### EXAMPLE 1

### Consumption of a Moensin-Containing Food Composition

Consumption of the food composition of the present disclosure can be evaluated in bovines. Three groups of bovines can be evaluated in the present example. In one group, the food composition of the present disclosure containing monensin, a solvent, and an animal feed can be combined with a mineral mix fed to bovines. In a second group, a solid formulation of monensin can be combined with a mineral mix and an animal feed and fed to bovines. In a third group, a mineral mix and an animal feed can be combined as a control formulation and fed to bovines. The average intake of the combination (grams per day) for each group can be evaluated, as well as the average daily weight gain (kilograms per day). Table 1 shows the results of the study. Bovines fed the food composition of the present disclosure containing monensin, a solvent, and an animal feed can display a numerically superior intake of the combination and a greater intake compared to bovines fed the solid formulation of monensin combined with a mineral mix and an animal feed.

TABLE 1

| | | | Weekly Mineral |
|---|---|---|---|
| | | | Intake (g of |
| | Average Daily | Mineral Intake | mineral/kg of |
| Group | Gain (kg/day) | (g/day) | bodyweight) |
| Control | −0.04 | 71.5 | 1.18 |
| Food Composition | −0.16 | 56.0 (−22%)* | 0.92* |
| Prepared with | | | |
| Solid Formulation | | | |
| of Monensin | | | |
| Food Composition | −0.01 | 64.7 (−10%) | 1.07 |
| Prepared with | | | |
| Soluble Formulation | | | |
| of Monensin | | | |

*= p < 0.05 vs. control

1-42. (canceled)

43. A process for the preparation of an animal food composition comprising monensin, the process comprising combining an animal feed supplement in solution form which comprises monensin present in said animal feed supplement in a concentration of about 5% to about 40% (weight/weight), and a solvent selected from benzyl alcohol, oleic acid, propylene glycol, vitamin E, ethanol, glyceryl mono-caprylate, and di-caprylate, and mixtures thereof, wherein said solvent is present in said animal feed supplement in a concentration of about 60% to about 95% (weight/weight); with an animal feed, such that the monensin is present in said animal food composition in an amount therapeutic to said animal.

44. The process of claim 43, wherein monensin is present at a concentration of approximately 15% (weight/weight) of said animal feed supplement.

45. The process of claim 43, wherein said solvent is present at a concentration of approximately 85% (weight/weight) of said animal feed supplement.

46. The process of claim 43, wherein said process further comprises the step of allowing the solvent to evaporate such that said solvent is present in said animal food composition in a residual amount.

47. The process of claim 43, wherein said animal food composition comprises an amount of monensin therein whereby said animal consumes monensin in an amount of about 50 to about 500 mg/day.

48. An animal food composition prepared by the process of claim 43.

49. An animal food composition prepared by the process of claim 46.

50. The animal food composition of claim 48, wherein said animal food composition comprises an amount of monensin therein whereby said animal consumes monensin in an amount of about 50 to about 500 mg/day.

51. The animal food composition of claim 49, wherein said animal food composition comprises an amount of monensin therein whereby said animal consumes monensin in an amount of about 50 to about 500 mg/day.

52. A method of improving feed efficiency comprising administering the animal food composition of claim 48 to said animal.

53. The method of claim 52, wherein said animal is a bovine.

54. The method of claim 52, wherein said animal consumes monensin in an amount of about 50 to about 500 mg/day from the administration of said animal food composition.

55. A method of improving feed efficiency comprising administering the animal food composition of claim 49 to said animal.

56. The method of claim 55, wherein said animal is a bovine.

57. The method of claim 55, wherein said animal consumes monensin in an amount of about 50 to about 500 mg/day from the administration of said animal food composition.

58. A method of preventing or treating coccidiosis in an animal comprising administering the animal food composition of claim 48 to said animal.

59. The method of claim 58, wherein said animal is a bovine.

60. The method of claim 58, wherein said animal consumes monensin in an amount of about 50 to about 500 mg/day from the administration of said animal food composition.

61. A method of preventing or treating coccidiosis in an animal comprising administering the animal food composition of claim 49 to said animal.

62. The method of claim 61, wherein said animal is a bovine.

63. The method of claim 61, wherein said animal consumes monensin in an amount of about 50 to about 500 mg/day from the administration of said animal food composition.
